(19) United States
(12) Patent Application Publication
(10) Pub. No.: US 2026/0294235 A1
(43) Pub. Date: Oct. 1, 2026

(54) SYSTEMS, APPARATUS AND METHODS FOR TREATMENT OF RETINAL AND MACULAR DISEASES USING ARTIFICIAL INTELLIGENCE

(71) Applicant: John Gregory LADAS, Germantown, MD (US)

(72) Inventor: John Gregory LADAS, Germantown, MD (US)

(21) Appl. No.: 19/482,388

(22) PCT Filed: May 7, 2024
(86) PCT No.: PCT/US2024/028165

§ 371 Date: Nov. 7, 2025

Related U.S. Application Data
(60) Provisional application No. 63/500,842, filed on May 8, 2023.

(51) Int. Cl. A61B 3/10; G06N 20/20; G16H 10/60; G16H 20/17

(52) U.S. Cl. CPC A61B 3/102; G06N 20/20; G16H 10/60; G16H 20/17

(57) ABSTRACT

An optical coherence tomography (OCT) device includes artificial intelligence for recommending a treatment plan for a patient with a retinal or macular disease such as age-related macular degeneration (AMD). The OCT device includes a sensor configured to quantify an initial level of macular edema or retinal exudation. The OCT device receives treatment information for a series of anti-vascular endothelial growth factor (anti-VEGF) injections to the patient. The OCT device performs OCT on the patient subsequent to each anti-VEGF injection to determine subsequent levels of edema or retinal exudation. The OCT device collects a set of training data including: the initial and subsequent levels of edema or exudation, patient information, and treatment information. The OCT device applies the training data to a machine-learning model trained on training data for a plurality of patients to determine a treatment plan for the retinal or macular disease of the patient.

### CROSS-REFERENCE TO RELATED APPLICATIONS

This application claims priority to U.S. Patent Application No. 63/500,842 titled “SYSTEMS, APPARATUS AND METHODS FOR TREATMENT OF RETINAL AND MACULAR DISEASES USING ARTIFICIAL INTELLIGENCE,” filed May 8, 2023, which is assigned to the assignee hereof, and incorporated herein by reference in its entirety.

### FIELD OF THE INVENTION

Aspects of the present invention relate to systems, apparatuses, and methods for treatment of retinal and macular diseases using artificial intelligence.

### BACKGROUND

Retinal and macular diseases are leading causes of blindness worldwide with billions of dollars spent annually to treat these diseases. Retinal and macular diseases include, for example, age-related macular degeneration (AMD), Diabetic Macular Edema (DME), edema from Retinal Vein Occlusions (RVO), and myopic choroidal neovascularization (CNV). Retinal and macular diseases may also include exudation due to retinal vascular diseases.

The visual prognosis for retinal and macular diseases has improved over the past decade with the use of anti-vascular endothelial growth factor (anti-VEGF) injections that can either stabilize or improve the edema associated with retinal and macular diseases. There are different formulas of anti-VEGF medications that have different properties such as the efficacy in a particular patient, the potential duration of effect, and cost.

Although the optical coherence tomography (OCT) measurements and morphology are the main factors in determining the course of treatment, other factors that could be taken into consideration are changes in visual acuity, age, and other comorbidities.

### SUMMARY

Aspects of the present disclosure may include apparatuses and methods for generating a treatment plan for retinal and macular diseases.

In some aspects, the techniques described herein relate to a method of treating retinal and macular diseases, including: performing optical coherence tomography (OCT) on a patient to quantify an initial level of macular edema or retinal exudation; administering a series of anti-vascular endothelial growth factor (anti-VEGF) injections to the patient over a period of time; performing additional periodic OCT on the patient subsequent to each anti-VEGF injection to determine treatment response as assessed by subsequent levels of edema or retinal exudation; collecting a set of training data based on the patient, the training data including: the initial level of macular edema or retinal exudation, the subsequent levels of edema or retinal exudation, patient information, and treatment information; and applying the training data for the patient to a machine-learning model trained on a plurality of sets of training data for a plurality of patients to determine a treatment plan for the AMD of the patient after the series of anti-VEGF injections.

In some aspects, the techniques described herein relate to a method, further including training the machine-learning model on the plurality of sets of training data to select the treatment plan that optimizes a visual acuity of the patient.

In some aspects, the techniques described herein relate to a method, wherein the patient information includes at least: an age, a visual acuity, and an indication of presence or absence of one or more other diseases.

In some aspects, the techniques described herein relate to a method, wherein the machine-learning model is trained using a gradient boosting library to build a random forest model.

In some aspects, the techniques described herein relate to a method, wherein the initial level of macular edema is a thickness measured by the OCT or a fluid volume measured by the OCT.

In some aspects, the techniques described herein relate to a method, wherein the treatment information includes at least: a date of each anti-VEGF injection, a medication of each anti-VEGF injection, and a dose of each anti-VEGF injection.

In some aspects, the techniques described herein relate to a method, further including administering an anti-VEGF injection indicated by the treatment plan to the patient.

In some aspects, the retinal or macular disease is age-related macular degeneration (AMD).

In some aspects, the retinal or macular disease is one or more of the group consisting of: age-related macular degeneration (AMD), Diabetic Macular Edema (DME), edema from Retinal Vein Occlusions (RVO), and myopic choroidal neovascularization (CNV).

In some aspects, the techniques described herein relate to an optical coherence tomography (OCT) device, including: a sensor configured to detect a level of macular edema in an eye; a memory storing computer-executable instructions; and a processor coupled to the memory and configured to execute the computer-executable instructions to cause the OCT device to: perform optical coherence tomography (OCT) on a patient to quantify an initial level of macular edema; receive treatment information for a series of anti-vascular endothelial growth factor (Anti-VEGF) injections to the patient over a period of time; perform additional periodic OCT on the patient subsequent to each Anti-VEGF injection to determine subsequent levels of edema; collect a set of training data based on the patient, the training data including: the initial level of macular edema, the subsequent levels of edema, patient information, and treatment information; and apply the training data for the patient to a machine-learning model trained on a plurality of sets of training data for a plurality of patients to determine a treatment plan for the AMD of the patient after the series of anti-VEGF injections.

In some aspects, the techniques described herein relate to a OCT device, wherein the at least one processor is configured to execute the instructions to cause the OCT device to train the machine-learning model on the plurality of sets of training data to select the treatment plan that optimizes a visual acuity of the patient.

In some aspects, the techniques described herein relate to a OCT device, wherein the patient information includes at least: an age, a visual acuity, and an indication of presence or absence of one or more other diseases.

In some aspects, the techniques described herein relate to a OCT device, wherein the machine-learning model is trained using a gradient boosting library to model a random forest.

In some aspects, the techniques described herein relate to a OCT device, wherein the initial level of macular edema is a thickness of a retina measured by the OCT or a fluid volume measured by the OCT.

In some aspects, the techniques described herein relate to a OCT device, wherein the treatment information includes at least: a date of each anti-VEGF injection, a medication of each anti-VEGF injection, and a dose of each anti-VEGF injection.

In some aspects, the techniques described herein relate to a computer network including: a memory storing computer-executable instructions; and a processor coupled to the memory and configured to execute the computer-executable instructions to cause the processor to: receive, from a plurality of optical coherence tomography (OCT) devices, training data including, for each of a plurality of patients: an initial level of macular edema or retinal exudation, a subsequent level of macular edema or retinal exudation after each of a series of anti-vascular endothelial growth factor (Anti-VEGF) injections to the patient over a period of time, patient information, and treatment information; train the machine-learning model on the training data to select a treatment plan that optimizes a predicted visual acuity of a patient based on a patient data set including: an initial level of macular edema for the patient, a subsequent level of edema after each of an initial series of anti-VEGF injections to the patient over a period of time, patient medical information, and patient treatment information; and apply the patient data set for the patient to the machine-learning model to determine the treatment plan for the AMD of the patient after the initial series of anti-VEGF injections.

In some aspects, the techniques described herein relate to a computer network, wherein the patient medical information includes at least: an age, a visual acuity, and an indication of presence or absence of one or more other diseases.

In some aspects, the techniques described herein relate to a computer network, wherein the machine-learning model is trained using a gradient boosting library to build a random forest model.

In some aspects, the techniques described herein relate to a computer network, wherein the initial level of macular edema is a thickness measured by the OCT or a fluid volume measured by the OCT.

In some aspects, the techniques described herein relate to a computer network, wherein the treatment information includes at least: a date of each anti-VEGF injection, a medication of each anti-VEGF injection, and a dose of each anti-VEGF injection.

In some aspects, the techniques described herein relate to a computer network, further including a patient computer device, wherein the processor is further configured to output the treatment plan to the patient computer device.

Additional advantages and novel features relating to aspects of the present invention will be set forth in part in the description that follows, and in part will become more apparent to those skilled in the art upon examination of the following or upon learning by practice thereof.

### BRIEF DESCRIPTION OF THE FIGURES

In the drawings:

FIG. 1 is a diagram of an example Optical Coherence Tomography (OCT) system configured to recommend a treatment for age-related macular degeneration (AMD) based on a trained model, in accordance with aspects of the present disclosure.

FIG. 2 is a diagram of an example course of treatment for a patient using the OCT system of FIG. 1, in accordance with aspects of the present disclosure.

FIG. 3 is a diagram of an example computer system, in accordance with aspects of the present disclosure.

FIG. 4 is a diagram of an example communication system 400 usable in accordance with aspects of the present disclosure.

FIG. 5 is a flowchart illustrating an example method of providing a recommended treatment plan for AMD, in accordance with aspects of the present disclosure.

### DETAILED DESCRIPTION

Upon initial evaluation of a patient with suspected exudative AMD, the patient undergoes a thorough clinical exam to verify that the cause is related to the AMD and not another disease process. Once verified, the patient undergoes a macular Optical Coherence Tomography (OCT) to quantify the amount of edema. At this point, a patient will typically be started on one of the anti-vascular endothelial growth factor (anti-VEGF) medications for a total of three injections one month apart. Rarely, a determination is made to switch the medication in this initial period. OCT to quantify the macular edema is measured at each visit to assess any improvement or worsening. After the initial three injections, a decision is made to stop therapy, change medications, “treat and observe” or “treat and extend”. This decision is mostly determined by the amount of edema seen on an OCT at this particular visit. This pattern is continued over the remaining life of the patient with each individual physician using their own clinical judgement based on their knowledge and experience to choose the best treatment for one particular patient.

The current practices for treatment of exudative AMD may face several problems. First, the ongoing treatment of the patient may rely on the subjective clinical judgement of the individual physician, who is limited to personal experience and published studies regarding the treatment options. As the number of treatment options expands, it may be difficult for an individual physician to predict the results of the different treatment options. Second, the patient may change physicians, and a new physician may not have knowledge about the factors involved in previous treatment decisions.

Aspects of the present disclosure may include systems, apparatuses, and methods for treating age-related macular degeneration (AMD). An OCT device or network device connected thereto may be configured to utilize artificial intelligence to recommend a treatment for AMD of a patient based on a history of OCT measurements as well as patient information and treatment information. In an aspect, the artificial intelligence may include a machine-learning model trained on a plurality of sets of training data for a plurality of patients. For example, the OCT device may be communicatively connected to a network of other OCT devices that pool training data for training the machine-learning model. Additionally, the network may provide the recommended treatment to a connected device, such as a mobile device of a patient, to educate the patient about treatment options.

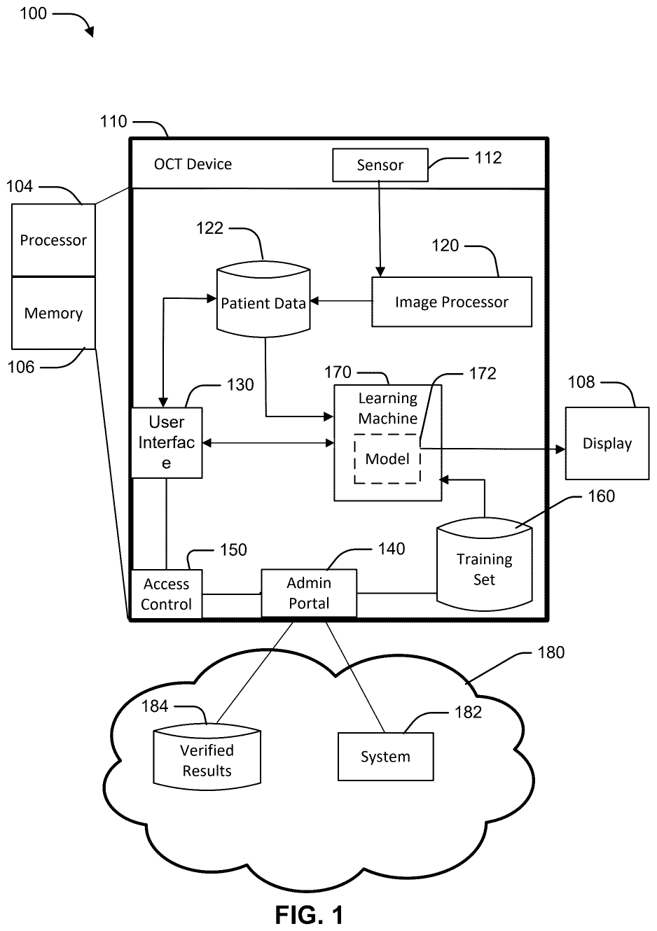

Turning now to FIG. 1, an example OCT system 100 may be configured to recommend a treatment for AMD based on a trained model 172. The OCT system 100 may be implemented on an OCT device 110 and associated computer. The OCT device 110 may include a sensor 112. For example, the sensor 112 may use reflected light waves to create an image of the back of an eye (e.g., the retina and optic nerve). The sensor 112 measures how much light is reflected by different layers of the retina and optic nerve. OCT can reveal the thickness, structure, and health of the retina and optic nerve. In some implementations, OCT images may be used to determine fluid volumes within the eye.

The OCT system 100 may further include a computer system. For example, the OCT system 100 may include a processor 104 and a memory 106. The processor 104 may store computer-executable instructions for performing the processes described herein. In some implementations, the computer system may be integrated with the OCT device. In some implementations, all or part of the computer system may be distributed. For example, in some implementations, the computer system may be a cloud network 180 including geographically distributed computing resources.

The OCT system 100 may include an image processor 120 that is configured to process the images from the sensor 112 to determine one or more properties of an eye. For example, the image processor 120 may analyze the images to determine a level of macular edema based on a measured thickness. As another example, the image processor 120 may determine a volume of fluid. The image processor may store the properties of the eye patient data in patient data storage 122.

The OCT system 100 may include a user interface 130. The user interface 130 may guide a user (e.g., a technician) through operating the OCT device 110 to obtain measurements for a patient. The user interface 130 may also include a user interfaces (e.g., graphical user interfaces) for the user to manually input patient data or obtain patient data from external sources such as electronic medical records. In some implementations, the user interface 130 may display patient data to the user, for example, by automatically importing measurements from the patient data storage 122 into fields of the user interface.

The OCT system 100 may include a learning machine 170 that is configured to train a model 172 for determining a treatment plan for AMD of a patient after a series of anti-VEGF treatments based on patient data and training data including AMD treatment information of a plurality of patients. The learning machine 170 may obtain the patient data from the patient data storage 122 and/or the user interface 130. The patient data may include: biographical information for the patient such as age, sex, and other medical conditions, an initial level of macular edema, an initial visual acuity, AMD treatment information such as date, medication, and dosage as well as pre-treatment or post-treatment level of macular edema and visual acuity. The learning machine 170 may obtain the training data from the training set 160, which may be aggregated based on patients examined using the OCT device 110 as well as external verified results 184. The training data may include the same types of data as the patient data.

The learning machine 170 may be a machine-learning library that provides tools for training the model 172. For example, the learning machine 170 may be a distributed gradient boosting library such as XGBoost. The gradient boosting library may be configurable to generate a recommendation system for recommending a treatment plan for an individual patient. The treatment plan recommended by the recommendation system may be the treatment plan that optimizes a predicted visual acuity of the individual patient based on the patient data. The distributed gradient boosting library may combine various machine-learning models such as decision trees, bagging classifiers, and random forests. Boosting refers to building a strong classifier from a number of weak classifiers by building subsequent models to correct for errors in previous models. Accordingly, multiple classifiers (e.g., decision trees) for recommending a treatment plan for the patient may be combined using a gradient boosting library to provide a stronger classifier. For example, each component classifier may be trained to classify the patient data into a component of the treatment plan such as recommended medication, dosage, and treatment interval. The weights of each component classifier may be selected to optimize the visual acuity based on the training data. The gradient boosting library may select among the component classifiers to optimize the visual acuity. Further, the model 172 may be validated using a reserved subset of the training data. Additional validation and/or retraining may be repeated using new sets of training data. The model 172 may output the recommended treatment plan to a display 108.

In another aspect, some functionality of the OCT system 100 may be performed via the cloud network 180. For example, the OCT system 100 may include an administrative portal 140 that controls communication between the OCT system 100 and the cloud network 180. The administrative portal 140 may be controlled locally via the user interface 130 and access control 150. For example, the access control 150 may allow certain users to retrieve and upload data to the cloud network 180.

The cloud network 180 may host various services that may be utilized by the OCT system 100. For example, the cloud network 180 may host a computing system 182 including computing resources such as processors and memory that may be used to execute some functionality described herein. For example, in some implementations, model training may be performed on the system 182. In some implementations, the cloud network 180 may include storage of verified results 184. The verified results 184 may be, for example, anonymized patient data that includes information regarding treatments for AMD such as OCT measurements, administered treatments, and results such as visual acuity. In some implementations, the OCT system 100 may import the verified results 184 to use as the training set 160.

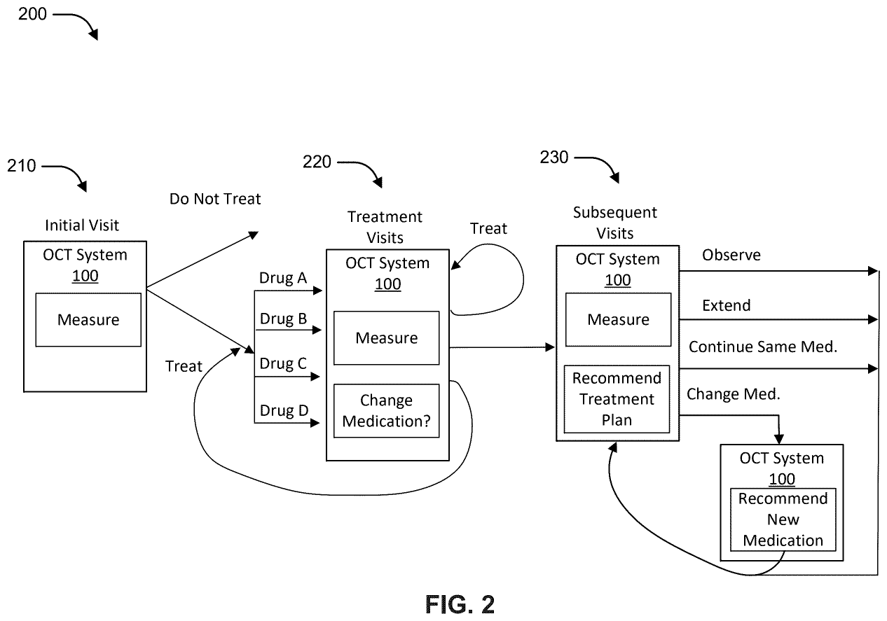

FIG. 2 is a diagram of an example course of treatment 200 for a patient using the OCT system 100. In an aspect, the OCT system 100 may perform OCT on the patient at each visit, which may be scheduled periodically (e.g., monthly). In an aspect, the course of treatment 200 may include an initial visit 210, treatment visits 220, and subsequent visits 230.

The initial visit 210 may be a visit with an ophthalmologist prior to a diagnosis of AMD. At the initial visit 210, the OCT system 100 may measure the patient's eyes. The measurements by the OCT system 100 may form part of an initial diagnosis. The initial diagnosis may be performed by the ophthalmologist. For example, the ophthalmologist may determine whether to treat AMD at the initial visit 210. The initial diagnosis may involve screening out conditions other than AMD for which the OCT device 110 and/or model 172 is not trained to diagnose. In some implementations, a set of patient data may not be available at an initial visit 210. For example, prior to any anti-VEGF treatment, the patient data may not include treatment information. In some implementations, where the ophthalmologist has diagnosed AMD, the ophthalmologist may further determine how to treat the AMD (e.g., which anti-VEGF medication to use). For instance, anti-VEGF medications may include AVASTIN (bevacizumab), LUCENTIS (ranibizumab), EYLEA (aflibercept), VABYSMO (faricimab-svoa), and BYOOVIZ (ranibizumab-nuna). Anti-VEGF medications may also include biosimilars to the example anti-VEGF medications. Once again, without past treatment information for the patient, the OCT device 110 and/or model 172 may not make a recommendation.

The treatment visits 220 may follow the initial visit 210 and a regular interval (e.g., 1 month) according to the treatment plan. Generally, the treatment plan will include three of more treatment visits 220. At treatment visits 220, the OCT system 100 may measure the patient's eyes, for example, by determining a thickness of the retina or a volume of fluid using the OCT device 110. Generally, the ophthalmologist may continue the treatment with the same medication according to the treatment plan. In some implementations, the OCT system 100 may provide a recommendation of whether to change a medication. For example, the model 172 may be able to detect, based on the measurements following the initial treatment, that there is a problem with the initially selected medication. Such issues may be relatively uncommon.

The subsequent visits 230 may occur after the treatment visits 220. The subsequent visits 230 may including measuring the eyes of the patient using the OCT device 110. In an aspect, treatment plans for patients may diverge significantly after the treatment visits 220 based on the results from the treatment visits 220. Generally, the ophthalmologist may determine whether to observe the patient without further treatments, extend the treatments, continue the same medication, or change medications. In an aspect, the OCT system 100 may make a recommendation for the treatment plan of the patient. For example, the OCT system 100 may apply the training data for the patient to the model 172 to determine a treatment plan for the AMD of the patient after the series of anti-VEGF injections based on the treatment information regarding the treatments at the treatment visits along with the measurements of the patient's eyes and patient information. The recommended treatment plan may include a course of action such as: observe the patient without further treatments, extend the treatments, continue the same medication, or change medications. In some implementations, where the recommendation is to change a medication, the OCT system 100 may make a second recommendation regarding the medication. For example, in some implementations, the second recommendation may include a medication, a dosage for the medication, and frequency of the treatments. In some implementations, the OCT system 100 may include a first model that recommends a treatment plan and a second model that recommends a specific treatment plan for a new medication.

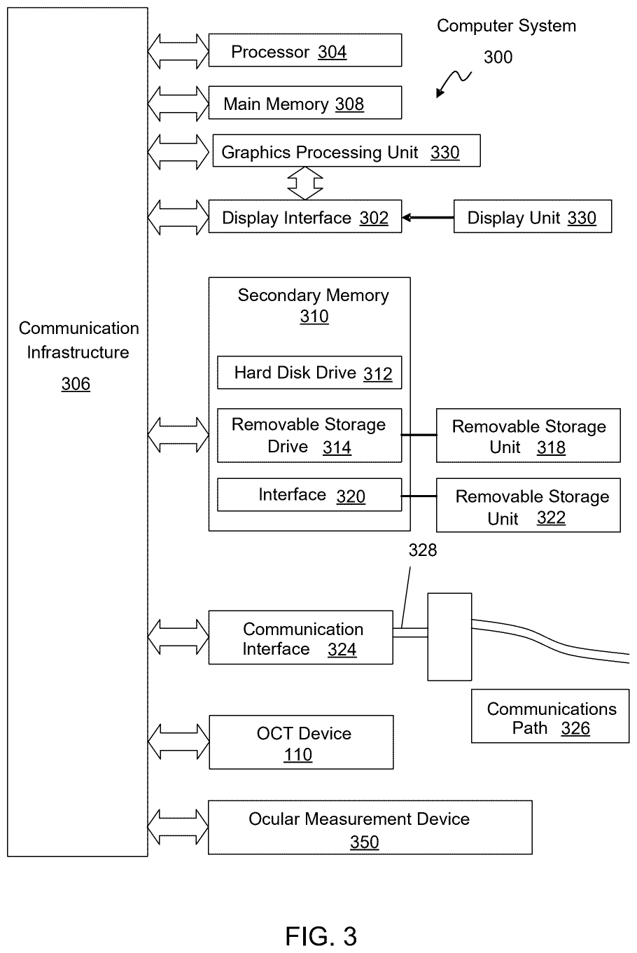

Aspects of the present disclosure may be implemented using hardware, software, or a combination thereof and may be implemented in one or more computer systems or other processing systems. In an aspect of the present disclosure, features are directed toward one or more computer systems capable of carrying out the functionality described herein. An example of such a computer system 300 is shown in FIG. 3.

Computer system 300 includes one or more processors, such as processor 304. The processor 304 is connected to a communication infrastructure 306 (e.g., a communications bus, cross-over bar, or network). Various software aspects are described in terms of this example computer system. After reading this description, it will become apparent to a person skilled in the relevant art(s) how to implement aspects of the disclosure using other computer systems and/or architectures.

Computer system 300 can include a display interface 302 that forwards graphics, text, and other data from the communication infrastructure 306 (or from a graphics processing unit (GPU) 332) for display on a display unit 330. For example, the display interface 302 may forward a graphical rendering of a super surface from the processor 304 to the display unit 330. Computer system 300 also includes a main memory 308, preferably random access memory (RAM), and may also include a secondary memory 310. The secondary memory 310 may include, for example, a hard disk drive 312 and/or a removable storage drive 314, representing a floppy disk drive, a magnetic tape drive, an optical disk drive, a universal serial bus (USB) flash drive, etc. The removable storage drive 314 reads from and/or writes to a removable storage unit 318 in a well-known manner. Removable storage unit 318 represents a floppy disk, magnetic tape, optical disk, USB flash drive, etc., which is read by and written to removable storage drive 314. As will be appreciated, the removable storage unit 318 includes a computer usable storage medium having stored therein computer software and/or data.

Alternative aspects of the present disclosure may include secondary memory 310 and may include other similar devices for allowing computer programs or other instructions to be loaded into computer system 300. Such devices may include, for example, a removable storage unit 322 and an interface 320. Examples of such may include a program cartridge and cartridge interface (such as that found in video game devices), a removable memory chip (such as an erasable programmable read only memory (EPROM), or programmable read only memory (PROM)) and associated socket, and other removable storage units 322 and interfaces 320, which allow software and data to be transferred from the removable storage unit 322 to computer system 300.

Computer system 300 may also include a communications interface 324. Communications interface 324 allows software and data to be transferred between computer system 300 and external devices. Examples of communications interface 324 may include a modem, a network interface (such as an Ethernet card), a communications port, a Personal Computer Memory Card International Association (PCMCIA) slot and card, etc. Software and data transferred via communications interface 324 are in the form of signals 328, which may be electronic, electromagnetic, optical or other signals capable of being received by communications interface 324. These signals 328 are provided to communications interface 324 via a communications path (e.g., channel) 326. This path 326 carries signals 328 and may be implemented using wire or cable, fiber optics, a telephone line, a cellular link, a radio frequency (RF) link and/or other communications channels.

Accordingly, in one or more example implementations, the functions described may be implemented in hardware, software, or any combination thereof. If implemented in software, the functions may be stored on or encoded as one or more instructions or code on a computer-readable medium. Computer-readable media includes computer storage media, which may be referred to as non-transitory computer-readable media. Non-transitory computer-readable media may exclude transitory signals. Storage media may be any available media that can be accessed by a computer. By way of example, and not limitation, such computer-readable media can include a random-access memory (RAM), a read-only memory (ROM), an electrically erasable programmable ROM (EEPROM), optical disk storage, magnetic disk storage, other magnetic storage devices, combinations of the aforementioned types of computer-readable media, or any other medium that can be used to store computer executable code in the form of instructions or data structures that can be accessed by a computer.

In an aspect, the computer system 300 may include the OCT device 110 and/or another ocular measurement device 350. The OCT device 110 may determine one or more ocular measurement parameters such as a thickness of the retina or a volume of liquid. In some implementations, the OCT device 110 may include or be associated with another ocular measurement device 350, which may include any device for measuring an eye. For example, the ocular measurement device may include a biometer configured to measure an axial length and a corneal power of an eye. In an aspect, the ocular measurement device may further measure a white-to-white distance, anterior chamber depth, pre-operative refraction, and/or lens thickness. The ocular measurement device 350 may further receive input of ocular measurement parameters (e.g., gender or sex). The axial length may be a distance from the surface of the cornea to the retina. The corneal power may be a dioptric power of the cornea. As another example, the ocular measurement device 350 may measure an anterior chamber depth of an eye. In an aspect, the ocular measurement device 350 may be an ultrasound device. In another aspect, the ocular measurement device 350 may be an optical biometer. Various optical biometers are available under the names LENSTAR® and IOL MASTER. In another aspect, the ocular measurement device 350 may include an intraoperative abberrometry device. The intraoperative abberrometry device may take measurements of refractive properties of the eye during surgery. For example, an intraoperative abberrometry device may provide information on sphere, cylinder, and axis of the eye. Additionally, an ocular measurement device may include a measurement device such as a wavefront analyzer or autorefractor. The ocular measurement device 350 may be communicatively coupled to the processor 304 via the communication infrastructure 306, the communications interface 324, and/or the communications path 326.

Computer programs (also referred to as computer control logic) are stored in main memory 308 and/or secondary memory 310. Computer programs may also be received via communications interface 324. Such computer programs, when executed, enable the computer system 300 to perform the features in accordance with aspects of the present disclosure, as discussed herein. In particular, the computer programs, when executed, enable the processor 304 to perform the features in accordance with aspects of the present disclosure. Accordingly, such computer programs represent controllers of the computer system 300.

In an aspect of the present disclosure where the disclosure is implemented using software, the software may be stored in a computer program product and loaded into computer system 300 using removable storage drive 314, hard drive 312, or communications interface 320. The control logic (software), when executed by the processor 304, causes the processor 304 to perform the functions described herein. In another aspect of the present disclosure, the system is implemented primarily in hardware using, for example, hardware components, such as application specific integrated circuits (ASICs). Implementation of the hardware state machine so as to perform the functions described herein will be apparent to persons skilled in the relevant art(s).

In yet another aspect of the present disclosure, the disclosure may be implemented using a combination of both hardware and software.

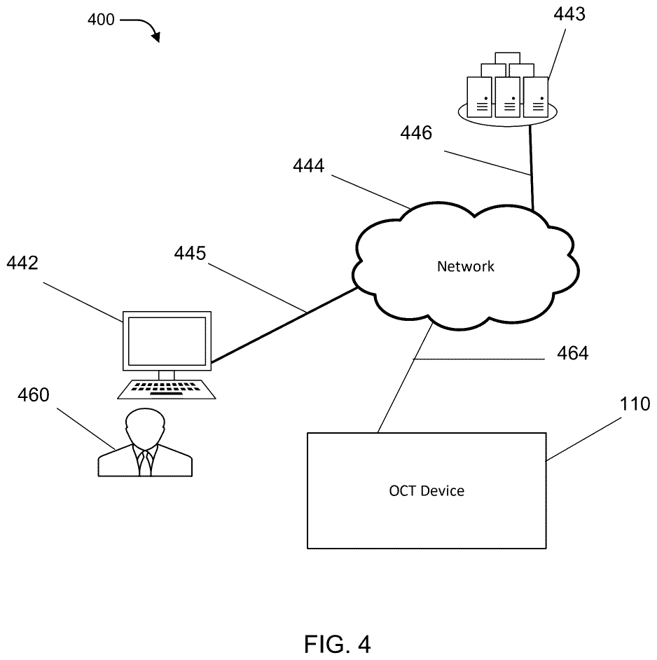

FIG. 4 shows a communication system 400 usable in accordance with aspects of the present disclosure. The communication system 400 includes one or more accessors 460 (also referred to interchangeably herein as one or more “users”) and one or more terminals 442 and/or other input device or devices (e.g., an OCT device 110). The OCT device 110 may further be configured to communicate with the network 444. In one aspect of the present disclosure, data for use is, for example, input and/or accessed after being received from an input device by accessors 460 via terminals 442, such as personal computers (PCs), minicomputers, mainframe computers, microcomputers, telephonic devices, or wireless devices, personal digital assistants (“PDAs”) or a hand-held wireless devices (e.g., wireless telephones) coupled to a server 443, such as a PC, minicomputer, mainframe computer, microcomputer, or other device having a processor and a repository for data and/or connection to a repository for data, via, for example, a network 444, such as the Internet or an intranet, and/or a wireless network, and couplings 445, 446, 464. The couplings 445, 446, 464 include, for example, wired, wireless, or fiberoptic links. In another aspect of the present disclosure, the method and system of the present disclosure may include one or more features that operate in a stand-alone environment, such as on a single terminal.

In an aspect, the server 443 may be an example of the computer system 300 (FIG. 3). In an aspect, for example, the server 443 may be configured to perform the methods described herein. For example, the server 443 may obtain measurements such as a retinal thickness or volume of fluid from the OCT device 110. The measurements may be entered by an accessor 460, or provided by the OCT device 110 (FIG. 3). The server 443 may also obtain other patient information including treatment information. The server 443 may generate a set of training data including patient information for a plurality of patients, possibly from different OCT devices 110. The server 443 may train a model 172 (FIG. 1) based on the training data. In some implementations, the server 443 may export the trained model to the OCT device 110 or another device such as a terminal 442. The server 443, OCT device 110, and/or terminal 442 may be configured to apply patient data to the machine-learning model 172 to determine a treatment plan for the AMD of the patient after the series of anti-VEGF injections.

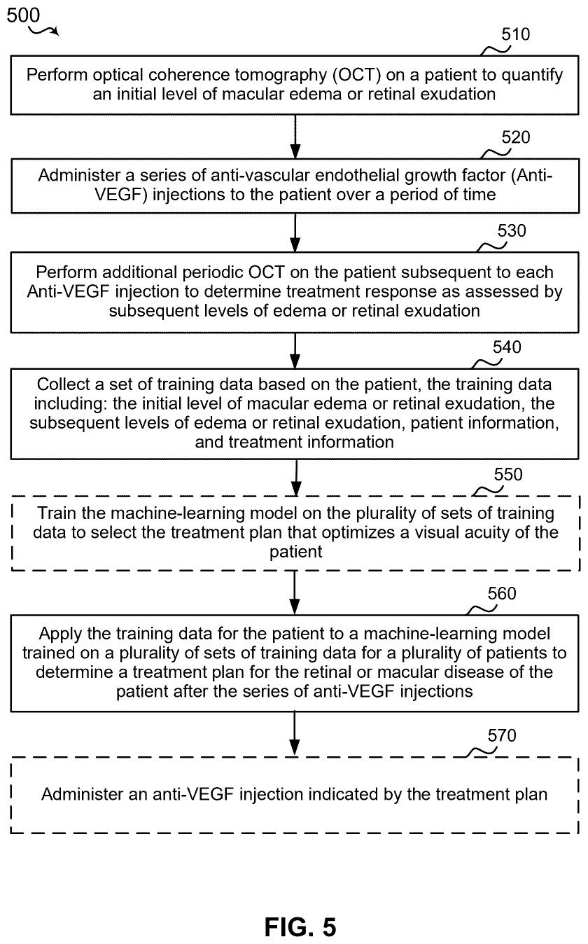

FIG. 5 is a flowchart illustrating an example method 500 of recommending a treatment plan for a patient with a retinal or macular disease. For example, the retinal or macular disease may be one or more of the group consisting of: age-related macular degeneration (AMD), Diabetic Macular Edema (DME), edema from Retinal Vein Occlusions (RVO), and myopic choroidal neovascularization (CNV). The method 500 may be performed by the system 100.

In block 510 the method 500 includes performing optical coherence tomography (OCT) on a patient to quantify an initial level of macular edema or retinal exudation. In an aspect, for example, the OCT device 110 may perform OCT on a patient using the sensor 112. The system 100 may use the image processor 120 to quantify an initial level of macular edema or retinal exudation. For example, the image processor 120 may analyze an image from the OCT device 110 to determine a thickness of the retina or a volume of fluid.

In block 520, the method 500 includes administering a series of anti-VEGF injections to the patient over a period of time. For example, an ophthalmologist may administer the series of anti-VEGF injections to the patient at the treatment visits 220. The ophthalmologist may record treatment information using the system 100. For example, the treatment information for the patient may include the date of the treatment, the anti-VEGF medication, and a dose of the treatment.

In block 530, the method 500 includes performing additional periodic OCT on the patient subsequent to each anti-VEGF injection to determine treatment response as assessed by subsequent levels of edema or retinal exudation. For example, the OCT device 110 may perform the additional periodic OCT on the patient at the next treatment visit 220 (e.g., prior to the next administration of anti-VEGF injection). The OCT device 110 may record the subsequent level of edema or retinal exudation.

In block 540, the method 500 includes collecting a set of training data based on the patient, the training data including: the initial level of macular edema, the subsequent levels of edema, patient information, and treatment information. In an aspect, for example, the OCT device 110 and/or the ophthalmologist may record the training data for a patient. For example, the OCT device 110 may record the initial level of macular edema or retinal exudation measured during the initial visit 210 and the subsequent levels of macular edema or retinal exudation measured at each treatment visit 220. The ophthalmologist may record the treatment information using the system 100. For example, the treatment information for the patient may include at least the date of the treatment, the anti-VEGF medication, and a dose of the treatment. The patient information may be entered by a user of the OCT system 100 such as the ophthalmologist, an assistant, or the patient, or the patient information may be acquired from an electronic medical record. For example, the patient information may include at least: an age, a visual acuity, and an indication of presence or absence of one or more other diseases.

In some implementations, in block 550, the method 500 optionally includes training the machine-learning model on the plurality of sets of training data to select the treatment plan that optimizes a visual acuity of the patient. For example, the learning machine 170 may execute a machine-learning algorithm such as a gradient boosting library to train the model 172. For instance, the model 172 may include a random forest model.

In block 560, the method 500 includes applying the training data for the patient to a machine-learning model trained on a plurality of sets of training data for a plurality of patients to determine a treatment plan for the retinal or macular disease of the patient after the series of anti-VEGF injections. In an aspect, for example, the system 100 may apply the training data for the patient to the machine-learning model 172. The machine-learning model 172 may provide a recommended treatment plan for the patient. For example, the recommended treatment plan may include one of continued observation, extending an existing treatment plan, continuing the same medication, or changing medication. In the case of changing medication, the recommended treatment plan may include the new medication, dose, and frequency of the treatments.

In block 570, the method 500 may optionally include administering an anti-VEGF injection indicated by the treatment plan. In an aspect, for example, the ophthalmologist may administer an anti-VEGF injection indicated by the treatment plan to the patient.

While aspects of the present disclosure have been described in connection with examples thereof, it will be understood by those skilled in the art that variations and modifications of the aspects of the present disclosure described above may be made without departing from the scope hereof. Other aspects will be apparent to those skilled in the art from a consideration of the specification or from a practice in accordance with aspects of the disclosure disclosed herein.

1. A method of treating retinal or macular disease, comprising:
performing optical coherence tomography (OCT) on a patient to quantify an initial level of macular edema or retinal exudation;
administering a series of anti-vascular endothelial growth factor (anti-VEGF) injections to the patient over a period of time;
performing additional periodic OCT on the patient subsequent to each anti-VEGF injection to determine treatment response as assessed by subsequent levels of macular edema or retinal exudation;
collecting a set of patient data based on the patient, the patient data including: the initial level of macular edema or retinal exudation, the subsequent levels of macular edema or retinal exudation, patient information, and treatment information; and
applying the patient data for the patient, including the subsequent levels of macular edema and the treatment information, to a machine-learning model trained on a plurality of sets of training data including patient data for a plurality of patients to determine a treatment plan for the retinal or macular disease of the patient after the series of anti-VEGF injections.

2. The method of claim 1, further comprising training the machine-learning model on the plurality of sets of training data to select the treatment plan that optimizes a visual acuity of the patient.

3. The method of claim 1, wherein the patient information includes at least: an age, a visual acuity, and an indication of presence or absence of one or more other diseases.

4. The method of claim 1, wherein the machine-learning model is trained using a gradient boosting library to build a random forest model.

5. The method of claim 1, wherein the initial level of macular edema or retinal exudation is a thickness of a retina measured by the OCT or a fluid volume measured by the OCT.

6. The method of claim 1, wherein the treatment information includes at least: a date of each anti-VEGF injection, a medication of each anti-VEGF injection, and a dose of each anti-VEGF injection.

7. The method of claim 1, further comprising administering an anti-VEGF injection indicated by the treatment plan to the patient.

8. The method of claim 1, wherein the retinal or macular disease is age-related macular degeneration (AMD).

9. The method of claim 1, wherein the retinal or macular disease is one or more of the group consisting of: age-related macular degeneration (AMD), Diabetic Macular Edema (DME), edema from Retinal Vein Occlusions (RVO), and myopic choroidal neovascularization (CNV).

10. An optical coherence tomography (OCT) device, comprising:
a sensor configured to detect a level of macular edema or retinal exudation in an eye;
a memory storing computer-executable instructions; and
a processor coupled to the memory and configured to execute the computer executable instructions to cause the OCT device to:
perform optical coherence tomography (OCT) on a patient to quantify an initial level of macular edema or retinal exudation of a patient having a retinal or macular disease;
receive treatment information for a series of anti-vascular endothelial growth factor (anti-VEGF) injections to the patient over a period of time;
perform additional periodic OCT on the patient subsequent to each anti-VEGF injection to determine treatment response as assessed by subsequent levels of macular edema or retinal exudation;
collect a set of patient data based on the patient, the patient data including: the initial level of macular edema or retinal exudation, the subsequent levels of macular edema or retinal exudation, patient information, and treatment information; and
apply the patient data for the patient, including the subsequent levels of macular edema and the treatment information, to a machine-learning model trained on a plurality of sets of training data including patient for a plurality of patients to determine a treatment plan for the retinal or macular disease of the patient after the series of anti-VEGF injections.

11. The OCT device of claim 10, wherein the processor is configured to execute the instructions to cause the OCT device to train the machine-learning model on the plurality of sets of training data to select the treatment plan that optimizes a visual acuity of the patient.

12. The OCT device of claim 10, wherein the patient information includes at least: an age, a visual acuity, and an indication of presence or absence of one or more other diseases.

13. The OCT device of claim 10, wherein the machine-learning model is trained using a gradient boosting library to model a random forest.

14. The OCT device of claim 10, wherein the initial level of macular edema or retinal exudation is a thickness of a retina measured by the OCT or a fluid volume measured by the OCT.

15. The OCT device of claim 10, wherein the treatment information includes at least: a date of each anti-VEGF injection, a medication of each anti-VEGF injection, and a dose of each anti-VEGF injection.

16. The OCT device of claim 10, wherein the retinal or macular disease is age-related macular degeneration (AMD).

17. The OCT device of claim 10, wherein the retinal or macular disease is one or more of the group consisting of: age-related macular degeneration (AMD), Diabetic Macular Edema (DME), edema from Retinal Vein Occlusions (RVO), and myopic choroidal neovascularization (CNV).

18. A computer network comprising:
a memory storing computer-executable instructions; and
a processor coupled to the memory and configured to execute the computer-executable instructions to cause the processor to:
receive, from a plurality of optical coherence tomography (OCT) devices, training data comprising, for each of a plurality of patients having a retinal or macular disease: an initial level of macular edema or retinal exudation, a subsequent level of macular edema or retinal exudation after each of a series of anti-vascular endothelial growth factor (Anti-VEGF) injections to the patient over a period of time, patient information, and treatment information;
train a machine-learning model on the training data to select a treatment plan that optimizes a predicted visual acuity of a patient based on a patient data set including: an initial level of macular edema for the patient, a subsequent level of edema after each of an initial series of anti-VEGF injections to the patient over a period of time, patient medical information, and patient treatment information; and
apply the patient data set for the patient, including the subsequent level of edema after each of an initial series of anti-VEGF injections and the patient treatment information, to the machine-learning model to determine the treatment plan for the retinal or macular disease of the patient after the initial series of anti-VEGF injections.

19. The computer network of claim 18, wherein the patient medical information includes at least: an age, a visual acuity, and an indication of presence or absence of one or more other diseases.

20. The computer network of claim 18, wherein the machine-learning model is trained using a gradient boosting library to build a random forest model.

21. (canceled)

22. (canceled)

23. (canceled)

24. (canceled)

25. (canceled)
